(19) United States
(12) Patent Application Publication
(10) Pub. No.: US 2026/0345970 A1
(43) Pub. Date: Nov. 5, 2026

(54) TRACKING GAMING ESTABLISHMENT MARKERS VIA PERMISSIONED BLOCKCHAIN

(71) Applicant: IGT, Las Vegas, NV (US)

(72) Inventors: Jeffery Shepherd, Reno, NV (US); Kevin Higgins, Reno, NV (US)

(21) Appl. No.: 19/198,989

(22) Filed: May 5, 2025

(51) Int. Cl. A61M 25/10

(52) U.S. Cl. CPC A61M 25/10184; A61M 2205/3337

(57) ABSTRACT

Systems and methods that following an activation of a gaming establishment marker from a first gaming establishment credit system, determine gaming establishment marker data associated with the activated gaming establishment marker. The systems and methods additionally cause the determined gaming establishment marker data to be written in a block of a permissioned blockchain accessible by a second, different gaming establishment credit system.

### BACKGROUND

Casinos are associated with multiple different channels of commerce including gaming activities (e.g., wagers on plays of games at electronic gaming machines and gaming tables) and non-gaming activities (e.g., making retail purchases at point-of-sale terminals throughout the casino).

### BRIEF DESCRIPTION OF THE SEVERAL VIEWS OF THE DRAWINGS

FIG. 1 is an example configuration of the architecture of a plurality of different components of different embodiments of the system of the present disclosure.

### DETAILED DESCRIPTION

In various embodiments, following an activation of a gaming establishment marker from a first gaming establishment credit system, the systems and methods of the present disclosure determine gaming establishment marker data associated with the activated gaming establishment marker. In these embodiments, following the activation of the gaming establishment marker from the first gaming establishment credit system, the systems and methods additionally cause the determined gaming establishment marker data to be written in a block of a permissioned blockchain accessible by a second, different gaming establishment credit system.

In certain embodiments, in association with an approval of a gaming establishment line of credit associated with a user, the system of the present disclosure employs a permissioned distributed ledger, such as a private blockchain, to report such an approval. In certain embodiments, in association with the activation of an amount of funds drawn against a gaming establishment marker associated with a user, the system of the present disclosure additionally or alternatively employs a permissioned distributed ledger, such as a private blockchain, to report such an activation. In certain embodiments, in association with a transfer of funds between a gaming establishment credit system and a gaming establishment account and/or a gaming establishment device, the system of the present disclosure additionally or alternatively employs a permissioned distributed ledger, such as a private blockchain, to report such a transfer. In these embodiments, given the availability of funds from a gaming establishment credit system and/or the flow of funds between the gaming establishment credit system and a gaming establishment fund management system and/or a gaming establishment device, the system writes, if permitted, data to a private blockchain to memorialize one or more transactions. Using such a private blockchain enables the gaming establishment credit system and/or other gaming establishment credit systems to have more information regarding a user's gaming establishment marker activity to make more informed determinations regarding future gaming establishment markers requested by that user.

More specifically, in association with the approval of a gaming establishment line of credit associated with a user and/or the activation of a gaming establishment marker associated with the user, the system employs a private blockchain to self-report changes to the user's gaming establishment credit profile. In such embodiments, to access the private blockchain, a component of the gaming establishment credit system first identifies itself to the private blockchain. Once the component of the gaming establishment credit system is validated, the gaming establishment credit system accesses the user's gaming establishment credit profile and updates the blockchain (e.g., writes to one or more blocks of the private blockchain) to reflect the approval of the line of credit and/or the activation of the gaming establishment marker. Such an update occurs independent of any central marker management entity and thus decentralizes how activities associated with gaming establishment markers are memorialized.

In addition to the gaming establishment credit system self-reporting changes to the user's gaming establishment credit profile independent of any central marker management entity, the system of the present disclosure enables other gaming establishment credit systems to utilize the updated credit profile of a user to enhance one or more determinations made by such other gaming establishment credit systems. In these embodiments, following a validation of a component of the other gaming establishment credit system, such a component accesses the user's gaming establishment credit profile written to the private blockchain to make one or more credit related decisions before offering additional credit to the user. Such access occurs independent of any central marker management entity and thus further decentralizes how activities associated with gaming establishment markers are retrieved amongst independently operating gaming establishment credit systems.

In certain instances, the system limits the level of access granted to one or more gaming establishment credit systems by limiting one or more of marker information (e.g., rather than providing an exact amount of outstanding markers associated with a user, the private blockchain returns a range of amounts), user information (e.g., the private blockchain anonymizes user data before making such user data accessible to certain gaming establishment credit systems) and/or entity information (e.g. the private blockchain does not identity the specific gaming establishment which issued the marker). Such a configuration enables the sharing of relevant credit profile data to enable different gaming establishment credit systems to make informed decisions on individually extending credit to the same user while ensuring that certain data (e.g., data used to market to an individual user) is not inappropriately shared between different entities. As such, the systems and methods of the present disclosure employ a permissioned distributed ledger, such as a private blockchain, to enable the access to gaming establishment credit data amongst a plurality of disparate entities which otherwise have no gaming establishment credit data sharing relationship with one another. In other words, to account for different entities individually tracking gaming establishment credit data associated with different users and further to minimize risks associated with different entities that are unaware of each other's credit activities extending gaming establishment credit to the same user, the system of the present disclosure utilizes a permissioned distributed ledger to enable the access and/or modification of such tracked gaming establishment credit data across such different entities. Accordingly, by associating different entities with the same distributed ledger and enabling such entities to utilize the distributed ledger to share part or all of the gaming establishment credit data sets which each respective entity individually maintains in association with an individual user, the system enables processes to be applied to different gaming establishment credit data sets across different entities to minimize risk and provided a more efficient operation of extending gaming establishment credit.

Such a configuration of using a private blockchain with limited access by different gaming establishment credit systems protects data of the individual gaming establishment credit systems while enabling each individual gaming establishment credit system the opportunity to harness the collective information of multiple gaming establishment credit systems. That is, rather than using a database that holds credit information about user's accessing gaming establishment credit and that further relies on a central marker management entity to act as the gatekeeper of that credit information to different gaming establishments that engage that central marker management entity, the employment of a decentralized private blockchain enables each of the gaming establishment credit systems at least limited access to such credit information independent of any central marker management entity. The minimization (or elimination) of the role of a central marker management entity represents a technical improvement by providing that a malfunction or other inoperable state of the central marker management entity does not cause a widespread outage of different gaming establishment credit system's ability to access data regarding one or more users. The systems of the present disclosure thus offer an operational improvement by broadening out gaming establishment credit data from being maintained by individual gaming establishment credit systems (which are prone to outages and lack the ability to share data) to being maintained by a private blockchain accessible, to different degrees, by multiple gaming establishment credit systems.

Accordingly, certain embodiments of the present disclosure enable an activation of a marker from a gaming establishment credit system and/or a transfer of funds from a gaming establishment credit system to a gaming establishment account (e.g., a cashless wagering account) and/or to a balance of a gaming establishment device (e.g., an electronic gaming machine) without the use of cash and with the employment of a permissioned blockchain to aid other gaming establishment credit system's future decisions regarding the user's credit worthiness. That is, since certain gaming establishment patrons are uncomfortable venturing into a gaming establishment with large amounts of cash, the systems enable a user access to an amount of funds from a gaming establishment marker without the need to handle cash and without the reliance on a central marker management entity. Such a reduction in the use of cash at a gaming establishment provides a relatively more secure environment for a user (via reducing or eliminating a user carrying cash on their person and thus diminishing the risks that such cash may be lost or stolen), overcomes various health concerns associated with cash-based transactions and cashless ticket voucher-based transactions (e.g., protecting patrons from using forms of currency and/or paper cashless ticket vouchers that act as transmission vehicles for contagions), and benefits the gaming establishment by reducing the use of certain kiosks that accept cash (e.g., reducing the wear and tear on such devices to prolong the operational life on these devices and/or saving gaming establishment processing resources and energy use dedicated to operating and maintaining such kiosks).

Additionally, by reducing the amount of cash transactions in a gaming establishment via enabling the use of gaming establishment markers while employing a permissioned blockchain to harness the collective knowledge of multiple gaming establishment credit systems, the systems reduce (or eliminate) human errors which often occur when cash is distributed at gaming establishment interfaces, such as casino desks and/or cashless ticket vouchers (e.g., an anonymous bearer instrument associated with an amount of funds and redeemable for cash via a kiosk, a cashier and/or game play on a gaming establishment device) are purchased with an amount of cash at gaming establishment interfaces, such as casino desks. Specifically, eliminating gaming establishment personnel from distributing cash and/or issuing ticket vouchers in exchange for amounts of cash not only protects the user (if the gaming establishment personnel provides the gaming establishment patron a lower amount of cash and/or a lower valued cashless ticket voucher than the patron deserves) but also protects the gaming establishment (if the gaming establishment personnel inadvertently or fraudulently provides the patron a higher amount of cash and/or or a higher valued cashless ticket voucher than the patron deserves). As such, the system of the present disclosure provides an alternative, non-cash-based and non-ticket voucher-based option for a gaming establishment patron to access funds at a gaming establishment device while utilizing a private blockchain to decentralize part or all of different users gaming establishment credit profiles.

In various embodiments, the present disclosure is directed to a system including or otherwise in communication with various components and/or sub-systems that operate together to cause a reporting to a private blockchain of an approval of a gaming establishment line of credit associated with a user, and/or an activation of an amount of funds drawn against a gaming establishment marker associated with a user, and/or an amount of funds transferred from a gaming establishment credit system to a gaming establishment device, such as an electronic gaming machine (with or without the employment of an intermediary gaming establishment account and independent of any cash or any cashless ticket vouchers). In these embodiments, the collection of components that are part of the system (or otherwise operate with one or more components of the system) facilitate private blockchain reportable credit transactions associated with a gaming establishment credit system. It should be appreciated that while generally described in association with employing a private blockchain to report to and retrieve from gaming establishment credit transactions occurring in association with a gaming establishment credit system, the employment of the private blockchain to report data to and/or retrieve data from extends to other gaming establishment systems (e.g., a fund management system, an anti-money laundering system, a financial transaction monitoring system, and/or a know-your-customer system) that benefit from reporting transactions to a private blockchain and/or retrieving transactions from the private blockchain.

In certain embodiments, the system includes or is otherwise in communication with one or more credit systems which each issue the user one or more lines of credit. Such a gaming establishment credit system facilitates the access of an amount of funds associated with a line of credit via one or more markers. These markers are accessible to enable a transfer of funds from the line of credit issued by the credit system to, either directly or indirectly through a gaming establishment account, a credit balance of a gaming establishment device, such as an electronic gaming machine (“EGM”) and/or a gaming table. In certain embodiments, the system also includes or is otherwise in communication with one or more credit reporting/credit risk systems. These credit reporting and risk systems monitor and report on a credit rating and status of one or more accounts maintained for the user at various funding sources, such as various financial institutions.

In various embodiments, the system enables a user to establish a marker with a gaming establishment credit system by applying for a line of credit (with or without presenting a check or other bank account information in the event a marker drawn against the line of credit is not paid back by the user) and the credit system approving the line of credit for up to a set amount of funds. In certain embodiments, to apply for a line of credit, the system enables a user to utilize an interface, such as a mobile device application being executed by a mobile device, a website accessed from a browser, a kiosk and/or a service window displayed by EGM (or other gaming establishment device), to attempt to complete the application through one or more interactive forms. For example, as part of applying for a line of credit, the user makes one or more inputs via an interface to provide certain user identifying information (such as, but not limited to, name, address, birthdate, state of birth, additional address details, a social security number and/or a mother's maiden name) and/or certain external account identifying information (such as, but not limited to, an identification of a funding source, and/or an external account number) in the event a marker drawn against the line of credit is not paid back by the user. Following the establishment of the line of credit, the system enables the user to utilize any suitable interface to view information about one or more available markers (e.g., an available amount of funds associated with the marker and/or an amount of funds associated with the marker previously accessed) as well as the balance of zero, one or more gaming establishment accounts.

In certain embodiments, as part of applying for a line of credit, the system determines zero, one or more security measures to invoke in association with one or more (or each) transaction that involves funds accessed via a marker. In these embodiments, to prevent unauthorized access to the funds associated with such a marker, the system applies such determined security measures in association with one or more (or each) transaction that attempts to access funds via the marker. In certain embodiments, as part of applying for a marker, the system determines one or more controls or restrictions to associate with the marker wherein if such controls are violated, the system invokes one or more security measures. In these embodiments, to balance the need to prevent unauthorized access to the funds associated with a marker against the need to provide a frictionless experience for users, the system dynamically employs one or more security measures such that certain transactions that trigger the need to employ enhanced security measures and certain transactions that do not trigger the need to employ enhanced security measures. It should be appreciated that an enhanced security measure includes any form of security that was not otherwise associated with the transaction prior to the determination that the nature of the transaction warranted an additional degree of protection to combat any attempted fraud associated with the transaction. For example, if a user is required to enter a personal identification number (“PIN”), provide a signature and/or present a biometric identifier for each attempted transfer of funds from a marker regardless of any determination that the nature of the transaction warrants any additional degree of protection to combat any attempted fraud associated with the transaction, such a PIN, signature and/or biometric identifier would not qualify as an enhanced security measure. In another example, if a user is not required to enter a PIN, provide a signature and/or present a biometric identifier for each attempted transfer of funds from a marker but following a determination that the nature of the transaction warrants requiring the user to enter a PIN, provide a signature and/or present a biometric identifier an as an additional degree of protection to combat any attempted fraud associated with the transaction, such a PIN, signature and/or biometric identifier would qualify as an enhanced security measure.

In certain embodiments, following the establishment of a line of credit accessible via one or more gaming establishment markers, the system enables the user to activate all or part of the available amount of funds. In these embodiments, the system enables the user to make one or more inputs regarding the requested access to a marker, such as, but not limited to, one or more inputs to indicate an amount of funds to be accessed via the marker, one or more inputs to approve the indicated amount of funds to be accessed via the marker, and/or one or more inputs to confirm the accessing of the marker. In certain embodiments, the gaming establishment credit system operates with a gaming establishment cashless system to transfer the amount of funds drawn against the gaming establishment marker to a cashless wagering account of the user maintained by the cashless wagering system and then to a gaming establishment device. In certain other embodiments, the gaming establishment credit system operates to transfer the requested amount of funds drawn against the gaming establishment marker directly to a gaming establishment device. In these embodiments, to request a transfer of an amount of funds via the marker directly or indirectly to a gaming establishment device, a user utilizes a physical instrument (e.g., a smart card or a user issued magnetic striped card which the user utilizes via inserting the card into a card reader associated with the gaming establishment device) to enable the user to identify themselves to the gaming establishment device (and the gaming establishment credit system) and interfaces with the gaming establishment device (or a component associated with the gaming establishment device) in association with the marker transaction. In certain embodiments, to request a transfer of an amount of funds via the marker directly or indirectly to the gaming establishment device, the system requires the user to pair a mobile device with the gaming establishment device. For example, to access an amount of funds via a marker, the system requires the user to pair a mobile device (which is executing a gaming establishment mobile device application) with a SMIB (i.e., a component of the gaming establishment patron management system) associated with the EGM and interface with at least the mobile device application to request an electronic transfer of funds from the gaming establishment credit system to the paired EGM.

In certain embodiments in which funds accessed from a gaming establishment marker are indirectly transferred to a gaming establishment device, following a receipt of user inputted data associated with a determined amount of funds to be accessed via the marker and any required confirmation from the user that they approve of the transaction, the gaming establishment credit system logs the user into the gaming establishment credit system (if necessary) and attempts to verify an activation of the amount of funds to be accessed via the marker of the user. Upon verifying the activation, the gaming establishment credit system proceeds with operating with the gaming establishment fund management system (e.g., a gaming establishment cashless wagering system) to log the user into a gaming establishment account, such as, but not limited to a cashless wagering account, associated with the user (if necessary) and potentially transfer the amount of funds to be accessed via the marker to the gaming establishment account associated with the user (e.g., to a gaming establishment cashless wagering account associated with the user.)

In operation of such embodiments, to complete a transfer of an amount of funds accessed via a marker to a gaming establishment account, the gaming establishment credit system determines whether to authorize the activation of the determined amount of funds. In these embodiments, if the gaming establishment credit system determines not to authorize the activation of the determined amount of funds via the marker, the gaming establishment credit system communicates one or more denial messages. In certain embodiments, following the denial, one or more display devices (e.g., display devices of a mobile device and/or the gaming establishment device) display a denial of marker activation to the user. On the other hand, if the gaming establishment credit system determines to authorize the activation of the determined amount of funds via the marker, the gaming establishment credit system updates the amount provided via the marker and communicates an authorization of the requested amount of funds to the gaming establishment fund management system. Upon receiving the authorization, the gaming establishment fund management system updates a balance of a gaming establishment account by the requested amount of funds activated via the marker. In certain embodiments, in association with the transfer, the system communicates a transfer of funds confirmation, wherein one or more display devices display a confirmation of the transfer of the amount of funds from the marker to the gaming establishment account.

In certain embodiments, following the completion of the transfer of an amount of funds associated with the marker to the gaming establishment account, such as a cashless wagering account associated with a user, the system enables the user to transfer an amount of funds from the gaming establishment account to a gaming establishment device. In certain embodiments, this transfer occurs in association with the gaming establishment device and/or a mobile device paired with (or otherwise operable to identify) the gaming establishment device. In certain other embodiments, following the completion of the transfer of an amount of funds associated with the marker to the gaming establishment account, such as a cashless wagering account associated with a user, the system automatically initiates a transfer of an amount of funds from the gaming establishment account to a gaming establishment device. In these embodiments, rather than the user making one or more inputs to attempt to transfer the amount of funds accessed via the marker to the gaming establishment device, the system automatically determines to transfer an amount of funds, such as the amount accessed via the marker, from the gaming establishment account to the gaming establishment device.

In these different embodiments, following a determination to transfer an amount of funds from the gaming establishment account to a gaming establishment device, a component of the gaming establishment fund management system, such as a server of the cashless wagering system, logs the user into a gaming establishment account associated with the user (if necessary) and requests the determined amount of funds to be transferred from the gaming establishment account to the gaming establishment device. In these embodiments, the component of the gaming establishment fund management system, such as a server of the cashless wagering system, determines whether to authorize the transfer of the amount of funds. If the component of the gaming establishment fund management system determines not to authorize the determined amount of funds, the component of the gaming establishment fund management system communicates a denial and, in certain embodiments, displays a denial of funds transfer to the user. On the other hand, if the component of the gaming establishment fund management system determines to authorize the amount of funds, the component of the gaming establishment fund management system updates the gaming establishment account associated with the user and communicates an authorization to the gaming establishment device. For example, when funds are transferred from a cashless wagering account to an EGM, the cashless wagering system reduces a balance of the cashless wagering account by the reduced amount of funds. The EGM (or a SMIB associated with the EGM) proceeds with updating a credit balance of the EGM to account for the amount of funds. In certain embodiments, the EGM further proceeds with communicating a transfer of funds confirmation and such a transferred amount of funds are then available for wagering at the EGM.

In certain embodiments in which funds accessed from a gaming establishment marker are directly transferred to a gaming establishment device, following receipt of the user inputted data associated with a determined amount of funds to be accessed via the marker and any required confirmation from the user that they approve of the transaction, the gaming establishment credit system logs the user into the gaming establishment credit system (if necessary) and attempts to verify an activation of the amount of funds to be accessed via the marker of the user. Upon verifying the activation, the gaming establishment credit system proceeds with operating with a gaming establishment device to transfer such funds to the gaming establishment device.

In operation of such embodiments, to complete a transfer of an amount of funds accessed via a marker to a gaming establishment account, the gaming establishment credit system determines whether to authorize the activation of the determined amount of funds. In these embodiments, if the gaming establishment credit system determines not to authorize the activation of the determined amount of funds via the marker, the gaming establishment credit system communicates one or more denial messages. In certain embodiments, following the denial, one or more display devices (e.g., display devices of a mobile device and/or the gaming establishment device) display a denial of marker activation to the user. On the other hand, if the gaming establishment credit system determines to authorize the activation of the determined amount of funds via the marker, the gaming establishment credit system updates the amount provided via the marker and initiates a transfer of an amount of funds to the gaming establishment device where the request originated from. For example, when funds drawn against a gaming establishment marker are transferred to an EGM, the gaming establishment credit system updates one or more databases to reflect the current debt associated with the gaming establishment marker and the EGM (or a SMIB associated with the EGM) proceeds with updating a credit balance of the EGM to account for the amount of funds (which are then available for wagering at the EGM).

It should be appreciated that in addition to transferring funds activated via a marker from a gaming establishment credit system directly or indirectly to a gaming establishment device, such as an EGM, the system enables a transfer of funds from a gaming establishment device (e.g., an EGM) and/or from a gaming establishment account (e.g., a cashless wagering account) to the gaming establishment credit system to payback or otherwise deactivate a marker. In certain embodiments, following the use of none, part or all of the transferred amount of funds at a gaming establishment device from an activated marker and upon receiving a “cash out” input from the user, part or all of the credit balance of the gaming establishment device is transferred directly to the gaming establishment credit system. In certain other embodiments, following the use of none, part or all of the transferred amount of funds at a gaming establishment device from an activated marker and upon receiving a “cash out” input from the user, part or all of the credit balance of the gaming establishment device is transferred indirectly to the gaming establishment credit system via an intermediate transfer to a gaming establishment account. In these embodiments, after a determination to transfer the credit balance of the gaming establishment to the gaming establishment credit system, the system proceeds with operating with the gaming establishment credit system (and, in certain instances, the gaming establishment fund management system) to log the user into the credit account (and in certain instances, the gaming establishment account, such as a cashless wagering account associated with the user) to execute the direct (or indirect) transfer of funds to the gaming establishment credit system to pay back the activated marker. Following such a transfer, the gaming establishment device proceeds with updating a credit balance of the gaming establishment device to account for the determined amount of funds transferred to pay back the activated marker and the gaming establishment credit system proceeds with updating the amount of funds outstanding drawn against the marker.

In certain embodiments, the system includes or is otherwise in communication with a marker reporting service operable with the gaming establishment credit system to coordinate the reporting of one or more markers to a distributed ledger (as described in more detail below). In these embodiments, the marker reporting service operates with the gaming establishment credit system to communicate marker data associated with one or more activated markers to one or more components of the distributed ledger. In these embodiments, the marker reporting service operates with the gaming establishment credit system to communicate marker data associated with one or more deactivated markers (i.e., one or more repaid markers) to one or more components of the distributed ledger. For example, the system includes one or more servers operating as a marker reporting service in communication with the gaming establishment credit system to enable certain actions, including, but not limited to, the reporting of data regarding marker activations and/or marker deactivations to one or more distributed ledgers.

In various embodiments, the system includes or is otherwise in communication with a gaming establishment fund management system that maintains one or more gaming establishment accounts. In certain such embodiments, the system includes or is otherwise associated with one or more cashless wagering systems. Each cashless wagering system is associated with or otherwise maintain one or more cashless wagering accounts. In certain embodiments, the system includes or in communication with one or more servers operating as a cashless wagering system that maintains a cashless wagering account and which is in communication with the gaming establishment credit system. In certain embodiments, the gaming establishment fund management system additionally operates with a gaming establishment device management system to direct funds from one or more gaming establishment accounts to the appropriate gaming establishment device.

In certain embodiments, a user utilizes a mobile device application running on a mobile device and/or a physical instrument (e.g., a smart card or a user issued magnetic striped card which the user utilizes via inserting the card into a component of a gaming establishment patron management system associated with a gaming establishment device) to facilitate the electronic transfer of any funds between a cashless wagering account (potentially transferred to the cashless wagering account from a gaming establishment credit system) and a gaming establishment device, such as a component of a gaming table and/or an EGM. In certain embodiments, a user utilizes a mobile device application running on a mobile device and/or a physical instrument (e.g., a smart card or a user issued magnetic striped card which the user utilizes via inserting the card into a kiosk) to facilitate the electronic transfer of any funds between a cashless wagering account (potentially transferred to the cashless wagering account from a gaming establishment credit system) to a credit balance accessible to wager on sporting events and/or a credit balance maintained remote from a gaming establishment device and utilized to wager on games of chance (or games of skill).

In certain embodiments, the system includes or is otherwise in communication with a gaming establishment device accounting system operable to monitor the occurrences of one or more financial transactions occurring in association with a gaming establishment. In certain such embodiments, the gaming establishment device accounting system operates with the gaming establishment credit system to monitor markers activated and tracks gaming establishment account balance movements in view of such activated markers.

In various embodiments, the system includes or is otherwise in communication with a payment gateway operable to interface with a banking network to communicate with one or more servers of one or more financial institutions that maintain one or more financial institution accounts and implement zero, one or more financial institution protocols, such as banking protocols. In such embodiments, the system includes a payment gateway operable with a banking network and one or more external funding sources (e.g., financial institutions) which maintain one or more external accounts (e.g., financial institution accounts) for a user to enable certain actions, including, but not limited to, balance inquiries, funding source enrollments and/or fund transfers. For example, the system includes one or more servers operating as a payment gateway in communication with a network of one or more servers of one or more banks or other financial institutions (i.e., the banking network and funding sources) which operate to electronically transfer funds from one or more accounts maintained at such banks or financial institutions to an EGM via a series of one or more intermediaries operating directly or indirectly with the payment gateway. In another example, the payment gateway is in communication with a mobile device which enables a user to conduct balance inquiries, request funding source enrollments and/or request fund transfers from one or more external funding sources. In different embodiments, the accounts of the external funding sources include, but are not limited to, one or more checking accounts maintained by one or more financial institutions (e.g., one or more banks and/or credit unions), one or more savings accounts maintained by one or more financial institutions, one or more financial institution accounts, such as a brokerage account, maintained by one or more financial institutions, one or more credit card accounts maintained by one or more financial institutions, one or more debit card accounts maintained by one or more financial institutions, and/or one or more third-party maintained accounts (e.g., one or more PayPal® accounts or Venmo® accounts).

In certain embodiments, the system includes or is otherwise in communication with an electronic funding service operable with the payment gateway. In these embodiments, the electronic funding service enables for a single entry point for all external funding transactions and if necessary, operates to disambiguate between destination sites of funds transferred from one or more external funding sources. For example, the system includes one or more servers operating as an electronic funding service in communication with one or more servers of the payment gateway to enable certain actions, including, but not limited to, balance inquiries, and/or fund transfers.

In certain embodiments, the system includes or is otherwise in communication with one or more gaming establishment patron management systems that, amongst other tasks, monitor activities at various points of contact associated with a gaming establishment and provides rewards, such as redeemable player tracking points, in association with such activities. In these embodiments, one or more components of the gaming establishment patron management system that are associated with an individual gaming establishment device operate to facilitate the transfer of funds from one or more gaming establishment accounts to that individual gaming establishment device. For example, the system employs a slot machine interface board (“SMIB”) (i.e., a component of a gaming establishment patron management system) associated with an EGM to enable certain actions, including, but not limited to, balance inquiries, marker activation reporting and/or fund transfers. In these embodiments, one or more components of the gaming establishment patron management system that are associated with an individual gaming establishment device also operate with a mobile device of a user to enable the component of the gaming establishment patron management system to identify, to at least the gaming establishment credit system, the gaming establishment device that will receive the funds from the gaming establishment credit system. For example, the system employs the SMIB associated with the EGM to pair with a mobile device that is in communication with the gaming establishment credit system to enable the gaming establishment credit system (and one or more intermediate components) to identify the appropriate EGM to transfer funds to.

In certain embodiments, the system utilizes one mobile device application to interact with the different components of the system to, amongst other actions, activate one or more markers, and/or transfer funds between one or more gaming establishment accounts and one or more gaming establishment devices. In certain embodiments, the system utilizes multiple mobile device applications to interact with the different components of the system to, amongst other actions, activate one or more markers, and/or transfer funds between one or more gaming establishment accounts and one or more gaming establishment devices. In certain of these embodiments, the mobile device applications include a location based digital wallet enabled application, such as a Passbook-enabled or Wallet-enabled application, which is accessible when the user enters a gaming establishment. In certain of such embodiments, the mobile device applications are downloaded to the mobile device from an application store. In certain of such embodiments, the mobile device applications are downloaded to the mobile device from one or more websites affiliated with the gaming establishment (which are accessible directly by the user and/or by a link opened when the user scans a QR code).

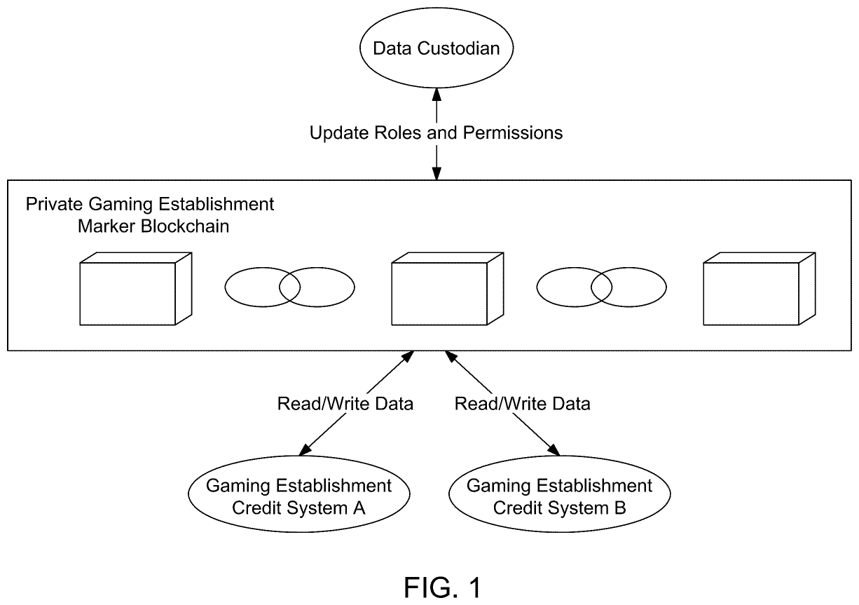

It should be appreciated that any system, sub-system or component of the present disclosure can be in communication with one or more other systems, sub-systems or components to facilitate, as appropriate, the activation one or more markers, a transfer of funds between one or more gaming establishment accounts and one or more gaming establishment devices and/or an interaction with a private blockchain to report on one or more of such transactions and/or retrieve data regarding one or more of such transactions. For example, while the gaming establishment credits systems of FIG. 1 are illustrated as being in communication with the private blockchain, in different embodiments, any system, sub-system or component of the present disclosure can be in communication with the private blockchain. Moreover, in certain embodiments, two or more of the systems, sub-systems and components of the present disclosure may be combined into a single system, sub-system or component. For example, while the gaming establishment credit system and the credit reporting/credit risk system are described as separate components, the gaming establishment credit system and the credit reporting/credit risk system may be combined into a single component operable to assess credit risk of a user and if appropriate, activate one or more markers for that user.

It should be appreciated that while described as a mobile device running a mobile device application being in communication with certain components of the present disclosure to, amongst other tasks, facilitate the approval of a gaming establishment marker, facilitate an activation of a gaming establishment marker, and/or facilitate the deactivation of a gaming establishment marker any suitable device, such as, but not limited to, a kiosk, a gaming establishment device (e.g., an interface of a gaming device, such as an EGM, or an interface of a non-gaming device, such as a retail point-of-sale terminal associated with a gaming establishment), an externally controlled interface displayed by a gaming establishment device (e.g., a remote host controlled service window displayed by an EGM), a component of a gaming establishment patron management system, such as a player tracking unit, and/or a gaming establishment interface, may be in communication with certain components of the present disclosure to, amongst other tasks, facilitate the approval of a gaming establishment marker, facilitate an activation of a gaming establishment marker, and/or facilitate the deactivation of a gaming establishment marker. It should be further appreciated that while described as funds activated from a gaming establishment marker are transferred (directly or indirectly through a gaming establishment account, such as a cashless wagering account) to an EGM (including, but not limited to, a slot machine, a video poker machine, a video lottery terminal, a terminal associated with an electronic table game, a terminal associated with a live table game, a video keno machine, a video bingo, and/or a sports betting terminal (that offers wagering games and/or sports betting opportunities)), in different embodiments, funds activated from a gaming establishment marker may be transferred to any suitable non-EGM gaming establishment device operable to receive funds, such as, but not limited to, a kiosk or a retail point-of-sale terminal associated with a gaming establishment.

In certain embodiments, the system enables activations of markers and/or transfers of funds associated with activated markers to different gaming establishment components to enable different gaming activities and/or non-gaming activities. In these embodiments, the system employs a service for interfacing with the various components to facilitate such actions. In certain embodiments, this service collects data from various components and utilizes such collected data to report to a distributed ledger and/or provide a singular view of the balances (or a plurality of singular views of different groupings of balances) available. In certain embodiments, such a service additionally or alternatively provides facilities to enforce rules and/or limitations including, but are not limited to, jurisdictional controls, self-imposed limits, state governmental controls and federal governmental controls, wherein the system provides the logic to determine how, and how much, to transfer to satisfy a request for funds while staying within the confines of such rules.

In certain embodiments, as mentioned above, the system operates with one or more distributed ledgers that store data associated with the credit extended to one or more users and/or the credit available to be extended to one or more users. These distributed ledgers enable the gaming establishment credit system to report the status of one or more accounts, such as credit accounts, maintained for one or more users. In other words, the employed distributed ledger enables gaming establishment credit systems to self-report data regarding users and query the ledger for information about users or potential users. These distributed ledgers further enable the gaming establishment credit system to retrieve the status of one or more accounts, such as credit accounts, maintained for one or more users. Put differently, the employed distributed ledger enables gaming establishment credit systems to query the ledger for information about users or potential users.

In certain embodiments, the distributed ledger takes the form of an external blockchain network that operates to electronically memorialize gaming establishment marker activities for different individual users. In these embodiments, the blockchain is a growing list of records, called blocks, that are cryptographically linked together such that each block relies on the data from the previous block and is resistant to modification absent having the proper keys. In certain such embodiments, the employed blockchain is a private or permissioned blockchain that it is not publicly available for anyone to access. Rather, given the sensitive user gaming establishment credit profile data reported to and maintained on the blockchain, the blockchain limits access to certain components of gaming establishment credit systems. Put differently, unlike public blockchains (e.g., a bitcoin blockchain) that enable anyone with the proper keys to transact with the blockchain, the permissioned or private blockchain employed restricts access to the blockchain (i.e., the records of the blockchain are not publicly accessible) such that users who are granted access are only enabled to perform the actions granted to them by a blockchain administrator. For example, as seen in FIG. 1, different gaming establishment credit systems associated with different entities interact with a private blockchain to read and/or write data regarding one or more markers activated in association with one or more users. It should be appreciated that while illustrated as employing a private blockchain to read and/or write data regarding one or more markers activated in association with one or more users, such a private blockchain may additionally or alternatively be employed to read and/or write data regarding different data sets employable by different gaming establishment credit systems such as, but not limited to, one or more marker defaults, one or more marker repayment transactions, one or more line of credit authorization openings, and/or one or more line of credit authorization closings.

Accordingly, in certain embodiments, following an activation of a gaming establishment marker from a first gaming establishment credit system, the system determines gaming establishment marker data associated with the activated gaming establishment marker, and causes the determined gaming establishment marker data to be written in a block of a permissioned blockchain accessible by a second, different gaming establishment credit system. Put differently, following an activation of a first gaming establishment marker from a first gaming establishment credit system, the system of certain embodiments causes data associated with the first gaming establishment marker to be written in a first block of a permissioned blockchain. In these embodiments, following an activation of a second, different gaming establishment marker from a second, different gaming establishment credit system, the system causes data associated with the second, different gaming establishment marker to be written in a second block of the permissioned blockchain.

It should be appreciated that depending on the configuration of the system and/or the blockchain, the modification of one or more attributes of a credit profile of a user on the blockchain may include updating the data associated with that credit profile to reflect the modified attribute or retiring that credit profile of the user and replacing the retired credit profile with a new credit profile for that user that retains certain unmodified attributes and the modified attributes. It should be further appreciated that one or more credit profiles of one or more users maintained on a distributed ledger of the present disclosure may be implemented in accordance with any suitable distributed ledger technology. Such distributed ledger technology which the credit profiles of the present disclosure may be associated with includes any suitable distributed ledger that enables recording and sharing of information across multiple data stores wherein each of the data stores (i.e., ledgers) includes the same data records, subject to maintenance and control through a distributed network of computing nodes. In different embodiments, such distributed ledger technology includes, but is not limited to, blockchain, directed acyclic graph, hashgraph, holochain, tempo (Radix), and any variation of these distributed ledger technologies in permissioned and/or permissionless form.

In certain embodiments wherein the employed distributed ledger technology utilizes blockchain technology, one or more credit profiles of one or more users are implemented in a blockchain, such as Ethereum. In these embodiments, the credit profile is either stored directly in the blockchain (e.g., using a smart contract that follows the ERC-721 standard) or a reference to the credit profile (e.g., a URL to the credit profile) is stored in the blockchain. In certain embodiments, various information associated with the created credit profile is indicated in the blockchain. In certain such embodiments, the information stored in the blockchain includes the credit data associated with the credit profile (e.g., an amount of an activated marker, when the amount of funds of the activated marker are due, an amount of a retired marker, when the retired marker was paid back), a URL associated with the credit profile, a history of the credit profile, and/or digital content associated with the credit profile. In other such embodiments, the information stored in the blockchain additionally (or alternatively) includes a state of the credit profile.

In certain embodiments wherein the employed distributed ledger technology differs from blockchain technology, one or more credit profiles are implemented in accordance with a directed acyclic graph protocol (which provides potentially higher throughput of transactions, and fee-less transactions). In these embodiments, the directed acyclic graph protocol enables the storing of the credit profile or a reference to the credit profile but without the utilization of blocks. Specifically, the directed acyclic graph of this embodiment employs a finite directed graph that includes an infinite amount of edges and vertices, wherein each edge is directed from one vertex to another without having to start at any one particular vertex or follow a consistent or directed sequence of edges to loop back to the same vertex again.

In operation of certain embodiments, in addition to enabling a user to borrow funds using a gaming establishment marker and retire funds borrowed using the gaming establishment marker, the system employs one or more distributed ledgers, such as a permissioned blockchain, to record such marker transactions. That is, in association with the approval of a gaming establishment line of credit, the activation of a gaming establishment marker associated with a user and/or the deactivation of a gaming establishment marker associated with the user, the system employs a permissioned blockchain to self-report changes to the user's gaming establishment credit profile. Such reporting enables other gaming establishment credit systems (which otherwise lacks the credit profile information of that user) to utilize the updated credit profile of the user to enhance one or more determinations made by such other gaming establishment credit systems. Put differently, a gaming establishment credit system posting, to a permissioned blockchain, a credit profile associated with a user enables other gaming establishment systems to make one or more credit related decisions before offering additional credit to the user.

In certain embodiments, prior to accessing a distributed ledger, such as the permissioned blockchain, maintained to aid gaming establishment credit systems in making informed decisions on offering credit to users, a participating entity associated with one more gaming establishment credit systems must first establish or otherwise become associated with (if needed) the permissioned blockchain by providing certain entity identifying information and/or certain permissioned blockchain identifying information, such as, but not limited to, a blockchain address, a public key and/or a source account identifier.

In certain embodiments, the participating entity provided access to the distributed ledger includes a gaming establishment credit system associated with an individual gaming establishment site. In certain other embodiments, the participating entity provided access to the distributed ledger includes a gaming establishment credit system associated with a plurality of gaming establishment sites. In these embodiments, one or more gaming establishment credit systems are associated with a corporate entity and various children entities (i.e., zero, one or more gaming establishment brands and zero, one or more gaming establishment venues having one or more EGMs and/or being associated with one or more personal gaming devices) that are associated with that corporate entity. In these embodiments, a gaming establishment venue includes a single property where an EGM may operate, a gaming establishment brand includes a group of gaming establishment venues under a single brand, and a corporate entity includes a group of one or more gaming establishment brands and/or a group of one or more gaming establishment venues. As such, zero, one or more gaming establishment credit systems provided access to the distributed ledger are associated with a single gaming establishment site and zero, one or more gaming establishment credit systems provided access to the distributed ledger are associated with a group of gaming establishment sites.

In certain embodiments, different participating entities associated with one or more gaming establishment credit systems have different levels of access to the data of the distributed ledger, such as the permissioned blockchain. In these embodiments, prior to providing an entity access, via the permissioned blockchain, to gaming establishment credit data associated with a user, the system determines zero, one or more permissions of that entity and the scope or magnitude of such determined permissions. For example, as seen in FIG. 1, a data custodian associated with the private blockchain determines (and periodically updates if warranted) the roles and permissions of how different gaming establishment credit systems may interact with (e.g., read data from and/or write data to) the private blockchain.

In certain embodiments, the system determines the scope of how the participating entity is permitted to interact with the distributed ledger by determining each entity's ability to access one or more aspects of the gaming establishment credit data stored on the distributed ledger. In these embodiments, the system determines which gaming establishment credit data stored on the distributed ledger each entity may view. In certain such embodiments, the system determines that an entity is enabled to access the distributed ledger to view a credit score for a user if the entity has paid for a lower initial tier of data access. In these embodiments, if the entity pays additional fees, the system determines that the entity is enabled to access the distributed ledger to view additional gaming establishment credit data associated with the user, such as the discrete credit amounts owed to other gaming establishment credit systems. In certain other embodiments, the system determines that an entity is enabled to access the distributed ledger based on being part of a grouping of entities that have agreed to interact together, using the distributed ledger, with respect to sharing gaming establishment credit data of one or more users. Such a configuration enables user to view any historical events associated with one or more markers, such as, but not limited to, being late on payment of any outstanding markers (e.g., any notices of default messages and/or notices of settlement for late payment).

In certain embodiments, the system determines the scope of how the participating entity is permitted to interact with the distributed ledger by determining each entity's ability to contribute one or more aspects of the gaming establishment credit data to be stored on the distributed ledger. In these embodiments, the system determines which gaming establishment credit data each entity may write to be stored on the distributed ledger.

In certain embodiments, the system determines the scope of how the participating entity is permissioned to interact with the distributed ledger by determining each entity's ability to modify one or more aspects of the gaming establishment credit data stored on the distributed ledger. In these embodiments, the system determines which gaming establishment credit data stored on the distributed ledger each entity may update or otherwise change. That is, in certain embodiments, a data custodian associated with the permissioned blockchain (e.g., an administrator of the permissioned blockchain) limits how zero, one or more entities access the blockchain and/or the type of records that can be added to and/or edited on the blockchain. In certain other embodiments, rather than enforcing permissions at the entity level, a data custodian associated with the permissioned blockchain (e.g., an administrator of the permissioned blockchain) limits all access to the blockchain and/or all types of records that can be added to and/or edited on the blockchain.

Accordingly, in certain embodiments, following data associated with a gaming establishment marker from a first gaming establishment credit system being written in a block of a permissioned blockchain, the system enables a second, different gaming establishment credit system access to the data associated with the gaming establishment marker written in the block of the permissioned blockchain, and disables a third, different gaming establishment credit system from accessing the data associated with the gaming establishment marker written in the block of the permissioned blockchain. In other words, following data associated with a gaming establishment marker from a first gaming establishment credit system being written in a block of a permissioned blockchain, the system of certain embodiments enables a second, different gaming establishment credit system with a first level of permission access to a first data set associated with the gaming establishment marker written in the block of the permissioned blockchain, and enables a third, different gaming establishment credit system with a second, different level of permission access to a second, different data set associated with the gaming establishment marker written in the block of the permissioned blockchain.

In certain embodiments, following a determination to post data associated with a gaming establishment marker transaction to a distributed ledger, such as the permissioned blockchain, maintained to aid gaming establishment credit systems in making informed decisions on offering credit to users and further following a determination that the entity to post the data is authorized to do so, a component of the gaming establishment credit system and/or a component of a marker reporting service creates and submits a transaction request to the distributed ledger. The transaction request includes (amongst other information) (1) a source of funds identifier, (2) a destination of funds identifier, (3) a transaction type identifier (i.e., drawing funds against a gaming establishment marker or paying back funds withdrawn against a gaming establishment marker), and/or (4) a transaction amount. In these embodiments, to cause a distributed ledger to store data associated with a gaming establishment marker transaction, the gaming establishment credit system and/or the marker reporting service provides (1) a source identifier (e.g., a gaming establishment credit account of a user which funds are being drawn against), (2) a destination identifier associated with where the funds are going (e.g., a cashless wagering account where the withdrawn funds are going), (3) a gaming establishment credit transaction event identifier, and/or (4) an amount of funds involved with the transaction.

In certain distributed ledgers, such as blockchain networks, accounts are identified by a public key associated with that account. For example, if a user has an account, then their account number is represented (or identified) by a public key (i.e., an RSA public key) that is tied to their account. The user keeps the corresponding private key (i.e., an RSA private key) secret and uses the corresponding private key to sign transaction requests submitted to the blockchain network. Accordingly, for the component of the gaming establishment credit system and/or the marker reporting service to trigger a memorialization of the marker transaction, the component of the gaming credit system and/or the marker reporting service provides a public key associated with the gaming establishment credit account of the user (and that represents the source or destination of the transaction request).

In certain embodiments, in reporting gaming establishment marker data to the private blockchain and/or retrieving gaming establishment marker data from the private blockchain, the gaming establishment credit system and/or the marker reporting service report and/or retrieve exact information associated with one or more gaming establishment markers. In these embodiments, the gaming establishment credit system and/or the marker reporting service report and/or retrieve detailed information such as, but not limited to, a user's total line of credit amount, an amount of funds outstanding on one or more gaming establishment markers, an amount of funds available (i.e., unused) on one or more lines of credit, a due date when an amount is owed, any amounts of funds repaid (i.e., retired) by the user, and/or any past due amounts.

In certain embodiments, in reporting gaming establishment marker data to the private blockchain and/or retrieving gaming establishment marker data from the private blockchain, the gaming establishment credit system and/or the marker reporting service report and/or retrieve general information associated with one or more gaming establishment markers. In these embodiments, to, amongst other reasons, prevent gaming establishment personnel from competing gaming establishments from employing the private blockchain to gain information regarding certain users which would otherwise not be available, the gaming establishment credit system and/or the marker reporting service report and/or retrieve less detailed information such as, but not limited to, a range of a user's total line of credit amount, a range of amounts of funds outstanding on one or more gaming establishment markers, a range of amounts of funds available (i.e., unused) on one or more lines of credit, a range of due dates when an amount is owed, a range of amounts of funds repaid by the user, and/or a range of any past due amounts. For example, if a user owes $25,000.00 to several casinos, a query of that user to the private blockchain returns that the user owes $20,000 to $30,000.

In certain embodiments, as mentioned above, the system enables permissioned entities to access the blockchain directly. In certain other embodiments, the system enables permissioned entities to access the blockchain through a software development kit provided by an administrator associated with the blockchain. In certain other embodiments, the system enables permissioned entities to access the blockchain via one or more application programming interfaces (and/or visual tools) provided by the administrator associated with the blockchain.

In certain embodiments, the component of the gaming establishment credit system and/or the marker reporting service provides information related to the user associated with the transaction request so that the marker transaction can later be accessed by the user responsive to the user providing that information. In certain of these embodiments, users can access the blockchain to determine their creditworthiness via browser-based pages for visual inspection of the data. In certain other embodiments, users can access information from the blockchain via one or more application programming interfaces that enable for programmatic inspection and decision making of, and on, the data. In certain embodiments, access to the gaming establishment credit account public key is restricted using gaming establishment credentials associated with the user (i.e., username/password information, a player tracking card and PIN, etc.). In these embodiments, once the user authenticates themselves using the gaming establishment credentials, the gaming establishment credit system and/or the marker reporting service enables the user to view their gaming establishment credit account public key and use that public key as an account identifier to include in the transaction request.

In certain embodiments, to enable the gaming establishment credit system and/or the marker reporting service to provide, to the distributed ledger, information related to the user, the gaming establishment credit system and/or the marker reporting service provide certain information that links the user to the gaming establishment credit account. In these embodiments, to prevent unauthorized postings to the distributed ledger in association with one or more gaming establishment marker transactions not associated with a particular user, the gaming establishment credit system and/or the marker reporting service provides information to verify the relationship between the user and the credit account. In certain such embodiments, the system employs certain information (e.g., random data) to include in the transaction request submitted to the blockchain network. For example, before any the marker transaction data is written to the private blockchain that tracks gaming establishment marker transactions, the system randomly generates information and associates the generated information with the user. In this example, the system employs the randomly generated information in verifying that the user is the owner of the credit account as part of a requested gaming establishment marker transaction associated with the user. In certain other embodiments, instead of providing information to include in the transaction request, the gaming establishment credit system and/or the marker reporting service verifies ownership of the corresponding private key associated with the public key of the credit account. For example, the system generates random information (e.g., a timestamp, nonce) that the gaming establishment credit system and/or the marker reporting service signs with the private key associated with the transaction request. In certain such embodiments, the gaming establishment credit system and/or the marker reporting service provides the signed data (i.e., the random information) for verification and to trigger a modification of the gaming establishment marker data stored by the blockchain network. Responsive to the system verifying the provided data (such as by comparing the signature of the provided data against a public key associated with the account that triggered the transfer), the system completes the update to the distributed ledger. However, if the system is unable to verify the provided data (e.g., if the signature of the provided data does not match the public key associated with the account), then the system does not update the distributed ledger.

In certain embodiments, since certain data on the private blockchain, such as personally identifiable information of one or more users, needs to be encrypted to meet various worldwide privacy regulations, or business data access restrictions, the gaming establishment credit system and/or the marker reporting service associated with one entity shares one or more encryption keys with the gaming establishment credit system and/or the marker reporting service associated with another entity. In these embodiments, since the system employs a private blockchain for different gaming establishment credit systems to share certain proprietary data, the system further employs a shared encryption key to enable one gaming establishment credit system and/or marker reporting service to decrypt data written to the blockchain by another gaming establishment credit system and/or marker reporting service.

It should be appreciated that given that the blockchain is a growing list of records, in certain embodiments, the private blockchain is indexed to enable each gaming establishment credit system and/or marker reporting service access to the most recent gaming establishment marker data stored within the blockchain for each user and each gaming establishment associated with the blockchain. In these embodiments, blockchain indexing enables for gaming establishment marker queries to run relatively faster and provide the most relatively relevant results given the dynamic nature of gaming establishment markers.

As described above, the employment of a permissioned blockchain enables multiple gaming establishment credit systems to monitor the credit history of a user (even if the identity of the user is unknown to a particular gaming establishment credit system) to make informed decisions regarding extending credit to the user. In certain embodiments, once the gaming establishment credit system and/or the marker reporting service is validated with the blockchain, the gaming establishment credit system and/or the marker reporting service can access the data on the blockchain and can query the blockchain for data related to the user. Such a query employs any data required to disambiguate a user from other users, such as one or more of an identification of a user with a gaming establishment patron management system, a name of the user, an address of the user, a partial social security number of the user, and/or a federated identification within the blockchain (which enables the gaming establishment credit system and/or the marker reporting service of one gaming establishment to use an identification of a user with a patron management system of that gaming establishment which would be different for that user at other gaming establishments).

In certain embodiments, to eliminate the risk of disclosing personally identifiable information of any individual users, the system employs a pre-defined algorithm in which certain personally identifiable information of a user is hashed in a specific order (e.g., social security number +first name +last name), and then all information associated with that user on the blockchain is associated with that value. In certain embodiments, to eliminate the risk of disclosing personally identifiable information of any individual users, the system additionally or alternatively employs a specific service that implements one or more pre-defined algorithms (which gaming establishment would not directly know it), to produce a token which is used to store data for a given user on the blockchain and/or used to search for data for the given user on the blockchain. In certain embodiments, to eliminate the risk of disclosing personally identifiable information of any individual users, the system additionally or alternatively employs one or more zero knowledge proofs in which records on the blockchain are recorded about user's marker history without clearly identifying the user. In these embodiments, when a first entity (e.g., a prover) wants to see if a given user has records on the blockchain, the first entity submits the request to a second entity (e.g., a verifier) which can validate the request from the first entity and find any associated records on the blockchain that may exist and return any details to the first entity as long as the details are correct. Such a valid zero knowledge proof further enables the first entity to decrypt detailed records of transactions for a given user.

In certain embodiments, the gaming establishment credit system and/or the marker reporting service employs one or more forms of anonymization in reporting the gaming establishment marker data to the blockchain and/or retrieving gaming establishment marker data from the blockchain. In these embodiments, to prevent gaming establishment personnel from one gaming establishment learning certain information about a user having no relationship with that gaming establishment (and thus posing a potential risk that gaming establishment personnel from another gaming establishment will try to use such information to establish a relationship with that user), certain gaming establishment marker data reported to the private blockchain and/or retrieved from the private blockchain is selectively anonymized. For example, if the gaming establishment credit system and/or the marker reporting service of Casino A is querying the private blockchain for user data, then user data related to Casino A is returned to Casino A and anonymized user data related to Casino B and Casino C is returned to Casino A. In this example, the anonymized user data related to Casino B and Casino C includes, but is not limited to, a name of one or more users (or other personally identifiable information of such users), the name of other casinos, the exact marker balance one or more users. As illustrated by this example, such a configuration prevents Casino A from learning any personally identifiable information about users from Casino B and Casino C whom have never visited Casino A (and thus prevents Casino A from taking advantage of data reported to the private blockchain to learn such personally identifiable information to market such users to visit Casino A).

In certain embodiments, in addition to the employment of a permissioned blockchain enabling multiple gaming establishment credit systems to monitor the credit history of a user (even if the identity of the user is unknown to a particular gaming establishment credit system) to make informed decisions regarding extending credit to the user, the employment of a permissioned blockchain enables multiple gaming establishment systems to monitor other activity of a user (even if the identity of the user is unknown to a particular gaming establishment system). That is, in addition to (or alternatively from) the permissioned blockchain including gaming establishment marker data, the permissioned blockchain includes additional (or alternative) data indicative of certain actions of a user that warrant being monitored. In certain such embodiments, the additional (or alternative) data indicative of certain actions of a user is written to the blockchain and accessible by any entity with the appropriate permission. In certain other embodiments, the additional (or alternative) data indicative of certain actions of a user is written to the blockchain and accessible by certain blockchain participants (e.g., channels in Hyperledger are used to enable the selective sharing of information amongst gaming establishments in a designated region).

In certain embodiments, the system additionally or alternatively employs the permissioned blockchain and one or more anti-money laundering services to ensure that certain activities occurring across one or more gaming establishments comply with various anti-money laundering obligations (e.g., anti-money laundering regulations) which require the tracking of certain financial transactions associated with a gaming establishment patron. In these embodiments, an anti-money laundering system associated with a gaming establishment reports to the permissioned blockchain potential money-laundering data associated with a user and other anti-money laundering systems associated with other gaming establishments retrieve, from the permissioned blockchain, such potential money-laundering data associated with the user to aid in one or more determinations regarding any money-laundering activities.

In operation of certain embodiments, an anti-money laundering system associated with a gaming establishment reviews (and/or triggers gaming establishment personnel to review) gaming session information communicated from a gaming establishment device (e.g., how many games were played, how the money got onto the gaming establishment device, if the money cashed out was actually played/wagered since it was deposited onto the gaming establishment device) to determine whether or not a user's interaction with the gaming establishment device has any characteristics indicative of an attempt to launder money. In these embodiments, if the anti-money laundering system determines that the user's interaction with the gaming establishment device has certain characteristics indicative of an attempt to launder money (e.g., the user has inserted relatively large sums of cash onto an EGM and then cashed out the money from the EGM with little gameplay), the anti-money laundering system reports such activity data to the permissioned blockchain. Once reported, the activity data is accessible by other gaming establishment anti-money laundering systems to aid in more quickly identifying (and potentially preventing before it occurs) money laundering activities associated with a particular user identified, in this example, by using a driver's license number, a full or partial social security number, or other personally identifiable information of that user.

Accordingly, in certain embodiments, the system determines if an activity of a user in association with a gaming establishment device of a first gaming establishment is associated with potential gaming establishment money laundering activity. In these embodiments, responsive to the determination being that the activity of the user is associated with potential gaming establishment money laundering activity, the system causes data associated with the activity to be written in a block of a permissioned blockchain. As such, following data associated with an activity of a user at a gaming establishment device of a first gaming establishment being written in a block of a permissioned blockchain as potential gaming establishment money laundering activity, the system of certain embodiments enables a second, different gaming establishment access to the data associated with the activity written in the block of the permissioned blockchain as potential gaming establishment money laundering activity, and disables an entity distinct from any gaming establishment from accessing the data associated with the activity written in the block of the permissioned blockchain as potential gaming establishment money laundering activity.

In certain embodiments, the system additionally or alternatively employs the permissioned blockchain and one or more Know Your Customer (“KYC”) services to validate user data across one or more gaming establishments. In these embodiments, a KYC system associated with a gaming establishment reports to the permissioned blockchain certain data associated with a user and other KYC systems associated with other gaming establishments retrieve, from the permissioned blockchain, such data associated with the user to aid in potentially validating the identity of the user. In operation of certain embodiments, a KYC system associated with a gaming establishment reviews (and/or triggers gaming establishment personnel to review) certain user data obtained and, if warranted, reports such data to the permissioned blockchain. Once reported, the user data is accessible by other gaming establishment KYC systems to aid in performing any KYC determinations and/or being aware of any potential issues associated with the user.

In certain embodiments, the system additionally or alternatively employs the permissioned blockchain and one or more financial transaction monitoring services to monitor certain financial activities occurring in association with multiple gaming establishments. In these embodiments, a financial transaction monitoring system associated with a gaming establishment reports to the permissioned blockchain certain financial transaction data associated with a user and other financial transaction monitoring systems associated with other gaming establishments retrieve, from the permissioned blockchain, such data associated with the user to aid in one or more determinations regarding how to proceed.

In certain such embodiments, a financial transaction monitoring system associated with a gaming establishment reviews (and/or triggers gaming establishment personnel to review) certain financial data associated with a user to determine whether or not such data is indicative of a fraudulent financial transaction (e.g., a user attempting to maliciously gain access to funds associated with a check from an account that lacks sufficient funds to cover the check). In these embodiments, if the financial transaction monitoring system determines that the user's activities are indicative of an attempt to engage in a fraudulent financial transaction, the financial transaction monitoring system reports such activity data to the permissioned blockchain. Once reported, the activity data is accessible by other gaming establishment financial transaction monitoring systems to aid in more quickly identifying (and potentially preventing before it occurs) such fraudulent financial transaction activities associated with a particular user identified, in this example, by using a driver's license number, a full or partial social security number, or other personally identifiable information of that user.

In certain other embodiments, a financial transaction monitoring system associated with a gaming establishment reviews certain financial data associated with a user and employs the private blockchain to obtain certain relevant financial data independent of any financial institution. In these embodiments, if a user at one gaming establishment attempts to cash a check issued by another gaming establishment (or otherwise obtain funds associated with another gaming establishment), the employment of the permissioned blockchain enables both gaming establishments to verify the validity of the check (or otherwise verify the funding instrument associated with the other gaming establishment). If the financial transaction monitoring system associated with either of the gaming establishments fails to verify the validity of the check using the permissioned blockchain, such gaming establishments operate to immediately change, without having to communicate with any financial institutions (e.g., banks) a state of the check (or other funding instrument) as invalid.

Accordingly, in certain embodiments, the system determines if an activity of a user in association with a first gaming establishment qualifies as a potential fraudulent financial transaction. In these embodiments, responsive to the determination being that the activity of the user qualifies as the potential fraudulent financial transaction, the system causes data associated with the activity to be written in a block of a permissioned blockchain. As such, following data associated with an activity of a user in association with a first gaming establishment being written in a block of a permissioned blockchain as a potential fraudulent financial transaction, the system of certain embodiments enables a second, different gaming establishment access to the data associated with the activity written in the block of the permissioned blockchain as the potential fraudulent financial transaction, and disables an entity distinct from any gaming establishment from accessing the data associated with the activity written in the block of the permissioned blockchain as the potential fraudulent financial transaction.

As such, the system and method of the present disclosure employs a permissioned distributed ledger, such as a private blockchain, to enable the access to gaming establishment credit data and/or gaming establishment non-credit data amongst a plurality of disparate entities which otherwise have no data sharing relationship with one another. In other words, to account for different entities individually tracking gaming establishment credit data and/or gaming establishment non-credit data associated with different users and further to minimize risks associated with different entities that are unaware of each other's activities related to the same user, the system of the present disclosure utilizes a permissioned distributed ledger to enable the access and/or modification of such tracked gaming establishment credit data and/or gaming establishment non-credit data across such different entities. Accordingly, by associating different entities with the same distributed ledger and enabling such entities to share (or even modify) part or all of the gaming establishment credit data sets and/or gaming establishment non-credit data sets which each respective entity individually maintains in association with an individual user, the system enables processes to be applied to different gaming establishment credit data sets and/or gaming establishment non-credit data sets across different entities to minimize risk and provide a more efficient operation of how gaming establishment systems interact with users.

Such a configuration of using a private blockchain with limited access by different gaming establishment systems protects data of the individual gaming establishment systems while enabling each individual gaming establishment system the opportunity to harness the collective information of multiple gaming establishment systems. That is, the employment of a decentralized private blockchain enables each of the gaming establishment systems at least limited access to credit information and/or non-credit information independent of any central management entity. The minimization (or elimination) of the role of a central management entity represents a technical improvement by providing that a malfunction or other inoperable state of the central management entity does not cause a widespread outage of different gaming establishment system's ability to access data regarding one or more users. The systems of the present disclosure thus offer an operational improvement by broadening out gaming establishment data from being maintained by individual gaming establishment systems (which are prone to outages and lack the ability to share data) to being maintained by a private blockchain accessible, in different degrees, by multiple gaming establishment systems.

It should be appreciated that the marker activation transfers of the present disclosure written to and/or retrieved from the private blockchain may occur in addition to or as an alternative from cash-based fund transfers and/or cashless ticket voucher-based fund transfers. It should be further appreciated that any functionality or process of the present disclosure may be implemented via one or more components of a gaming establishment system (e.g., a server of a gaming establishment credit system and/or a server of a gaming establishment fund management system), one or more gaming establishment devices (e.g., a gaming device such as an EGM or a gaming table component or a non-gaming device such as a gaming establishment personnel workstation), one or more components associated with a gaming establishment device (such as a SMIB or other component of a gaming establishment management system supported by or otherwise located inside the gaming establishment device), or a mobile device application. For example, while certain data or information of the present disclosure is explained as being communicated from a gaming establishment device, or a component associated with a gaming establishment device to a mobile device via one or more wireless communication protocols, such data or information may additionally or alternatively be communicated from one or more servers to a mobile device via one or more wireless communication protocols. Accordingly: (i) while certain functions, features or processes are described herein as being performed by a gaming establishment device, such functions, features or processes may alternatively be performed by one or more components associated with the gaming establishment device, one or more components of a gaming establishment system, or one or more mobile device applications, (ii) while certain functions, features or processes are described herein as being performed by a component associated with a gaming establishment device, such functions, features or processes may alternatively be performed by one or more gaming establishment devices, one or more components of a gaming establishment system, or one or more mobile device applications, (iii) while certain functions, features or processes are described herein as being performed by one or more mobile device applications, such functions, features or processes may alternatively be performed by one or more components of a gaming establishment system, one or more gaming establishment devices, or one or more components associated with a gaming establishment device, and/or (iv) while certain functions, features or processes are described herein as being performed by one or more components of a gaming establishment system, such functions, features or processes may alternatively be performed by one or more gaming establishment devices, one or more components associated with a gaming establishment device, or one or more mobile device applications.

In certain embodiments, the above-described embodiments of the present disclosure may be implemented in accordance with or in conjunction with zero, one or more gaming establishment devices (e.g., an EGM, or a gaming table component); zero, one or more components associated with a gaming establishment device (e.g., a SMIB associated with an EGM) and/or zero, one or more components of a gaming establishment system (e.g., a server of a gaming establishment credit system and/or a server of a gaming establishment fund management system). In these embodiments, such devices and components each include a controller including at least one processor.

The at least one processor is any suitable processing device or set of processing devices, such as a microprocessor, a microcontroller-based platform, a suitable integrated circuit, or one or more application-specific integrated circuits (ASICs), configured to execute software enabling various configuration and reconfiguration tasks, such as: (1) communicating with a remote source (such as a server that stores authentication information or fund information) via a communication interface of the controller; (2) converting signals read by an interface to a format corresponding to that used by software or memory of the gaming establishment device, the component associated with the gaming establishment device, and/or the component of the gaming establishment system; (3) accessing memory to configure or reconfigure parameters in the memory according to indicia read from the gaming establishment device, the component associated with the gaming establishment device, and/or the component of the gaming establishment system; (4) communicating with interfaces and the peripheral devices (such as input/output devices); and/or (5) controlling the peripheral devices. In certain embodiments, one or more components of the controller (such as the at least one processor) reside within a housing of the gaming establishment device, the component associated with the gaming establishment device, and/or the component of the gaming establishment system, while in other embodiments, at least one component of the controller resides outside of the housing of the gaming establishment device, the component associated with the gaming establishment device, and/or the component of the gaming establishment system.

The controller also includes at least one memory device, which includes: (1) volatile memory (e.g., RAM which can include non-volatile RAM, magnetic RAM, ferroelectric RAM, and any other suitable forms); (2) non-volatile memory (e.g., disk memory, FLASH memory, EPROMs, EEPROMs, memristor-based non-volatile solid-state memory, etc.); (3) unalterable memory (e.g., EPROMs); (4) read-only memory; and/or (5) a secondary memory storage device, such as a non-volatile memory device, configured to store software related information (the software related information and the memory may be used to store various files not currently being used and invoked in a configuration or reconfiguration). Any other suitable magnetic, optical, and/or semiconductor memory may operate in conjunction with the gaming establishment device, the component associated with the gaming establishment device, and/or the component of the gaming establishment system. In certain embodiments, the at least one memory device resides within the housing of the gaming establishment device, the component associated with the gaming establishment device, and/or the component of the gaming establishment system, while in other embodiments at least one component of the at least one memory device resides outside of the housing of the gaming establishment device, the component associated with the gaming establishment device, and/or the component of the gaming establishment system. In these embodiments, any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

The at least one memory device is configured to store, for example: (1) configuration software, such as all the parameters and settings on the gaming establishment device, the component associated with the gaming establishment device, and/or the component of the gaming establishment system; (2) associations between configuration indicia read from the gaming establishment device, the component associated with the gaming establishment device, and/or the component of the gaming establishment system with one or more parameters and settings; (3) communication protocols configured to enable the at least one processor to communicate with the peripheral devices; and/or (4) communication transport protocols (such as TCP/IP, USB, Firewire, IEEE 1394, Bluetooth, IEEE 802.11x (IEEE 802.11 standards), hiperlan/2, HomeRF, etc.) configured to enable the gaming establishment device, the component associated with the gaming establishment device, and/or the component of the gaming establishment system to communicate with local and non-local devices using such protocols. In one implementation, the controller communicates with other devices using a serial communication protocol. A few non-limiting examples of serial communication protocols that other devices, such as peripherals (e.g., a bill validator or a ticket printer), may use to communicate with the controller include USB, RS-232, and Netplex (a proprietary protocol developed by IGT).

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Aspects of the present disclosure are described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

In certain embodiments, the at least one memory device is configured to store program code and instructions executable by the at least one processor of the gaming establishment device, the component associated with the gaming establishment device, and/or the component of the gaming establishment system to control the gaming establishment device, the component associated with the gaming establishment device, and/or the component of the gaming establishment system. In various embodiments, part or all of the program code and/or the operating data described above is stored in at least one detachable or removable memory device including, but not limited to, a cartridge, a disk, a CD ROM, a DVD, a USB memory device, or any other suitable non-transitory computer readable medium. In certain such embodiments, an operator (such as a gaming establishment operator) and/or a user employs such a removable memory device in a component of the gaming establishment system to implement at least part of the present disclosure. In other embodiments, part or all of the program code and/or the operating data is downloaded to the at least one memory device of the gaming establishment device, the component associated with the gaming establishment device, and/or the component of the gaming establishment system through any suitable data network described above (such as an Internet or intranet).

The at least one memory device also stores a plurality of device drivers. Examples of different types of device drivers include device drivers for the gaming establishment device, the component associated with the gaming establishment device, and/or the component of the gaming establishment system and device drivers for the peripheral components. Typically, the device drivers utilize various communication protocols that enable communication with a particular physical device. The device driver abstracts the hardware implementation of that device. For example, a device driver may be written for each type of card reader that could potentially be connected to the gaming establishment device, the component associated with the gaming establishment device, and/or the component of the gaming establishment system. Non-limiting examples of communication protocols used to implement the device drivers include Netplex, USB, Serial, Ethernet, Firewire, I/O debouncer, direct memory map, serial, PCI, parallel, RF, Bluetooth™, near-field communications (e.g., using near-field magnetics), 802.11 (WiFi), etc. In one embodiment, when one type of a particular device is exchanged for another type of the particular device, the at least one processor of the gaming establishment device, the component associated with the gaming establishment device, and/or the component of the gaming establishment system loads the new device driver from the at least one memory device to enable communication with the new device. For instance, one type of card reader in the gaming establishment device, the component associated with the gaming establishment device, and/or the component of the gaming establishment system can be replaced with a second different type of card reader when device drivers for both card readers are stored in the at least one memory device.

In certain embodiments, the software units stored in the at least one memory device can be upgraded as needed. For instance, when the at least one memory device is a hard drive, new parameters, new settings for existing parameters, new settings for new parameters, new device drivers, and new communication protocols can be uploaded to the at least one memory device from the controller or from some other external device. As another example, when the at least one memory device includes a CD/DVD drive including a CD/DVD configured to store options, parameters, and settings, the software stored in the at least one memory device can be upgraded by replacing a first CD/DVD with a second CD/DVD. In yet another example, when the at least one memory device uses flash memory or EPROM units configured to store options, parameters, and settings, the software stored in the flash and/or EPROM memory units can be upgraded by replacing one or more memory units with new memory units that include the upgraded software. In another embodiment, one or more of the memory devices, such as the hard drive, may be employed in a software download process from a remote software server.

In some embodiments, the at least one memory device also stores authentication and/or validation components configured to authenticate/validate specified components of the gaming establishment device, the component associated with the gaming establishment device, and/or the component of the gaming establishment system and/or information, such as hardware components, software components, firmware components, peripheral device components, user input device components, information received from one or more user input devices, information stored in the at least one memory device, etc.

In certain embodiments, the peripheral devices include several device interfaces, such as, but not limited to: (1) at least one output device including at least one display device; (2) at least one input device (which may include contact and/or non-contact interfaces); (3) at least one transponder; (4) at least one wireless communication component; (5) at least one wired/wireless power distribution component; (6) at least one sensor; (7) at least one data preservation component; (8) at least one motion/gesture analysis and interpretation component; (9) at least one motion detection component; (10) at least one portable power source; (11) at least one geolocation module; (12) at least one user identification module; (13) at least one user/device tracking module; and (14) at least one information filtering module.

The at least one output device includes at least one display device configured to display any suitable information. In certain embodiments, the display devices are connected to or mounted on a housing of the gaming establishment device, the component associated with the gaming establishment device, and/or the component of the gaming establishment system. In various embodiments, the display devices serve as digital glass configured to aspects of the gaming establishment in which the gaming establishment device, the component associated with the gaming establishment device, and/or the component of the gaming establishment system is located. In various embodiments, the gaming establishment device, the component associated with the gaming establishment device, and/or the component of the gaming establishment system includes zero, one or more of the following display devices: (a) a central display device; (b) a player tracking display configured to display various information regarding a user's player tracking status; (c) a secondary or upper display device in addition to the central display device and the player tracking display; (d) a credit display configured to display a current quantity of credits, amount of cash, account balance, or the equivalent; and (e) a bet display. In various embodiments, the display devices include, without limitation: a monitor, a television display, a plasma display, a liquid crystal display (LCD), a display based on light emitting diodes (LEDs), a display based on a plurality of organic light-emitting diodes (OLEDs), a display based on polymer light-emitting diodes (PLEDs), a display based on a plurality of surface-conduction electron-emitters (SEDs), a display including a projected and/or reflected image, or any other suitable electronic device or display mechanism. In certain embodiments, as described above, the display device includes a touch-screen with an associated touch-screen controller. The display devices may be of any suitable sizes, shapes, and configurations.

In various embodiments, the at least one output device includes a payout device. In these embodiments, after the gaming establishment device, the component associated with the gaming establishment device, and/or the component of the gaming establishment system receives an actuation, the gaming establishment device, the component associated with the gaming establishment device, and/or the component of the gaming establishment system causes the payout device to provide a payment to the user. In certain embodiments, the payout device is one or more of: (a) a ticket printer and dispenser configured to print and dispense a ticket or credit slip associated with a monetary value, wherein the ticket or credit slip may be redeemed for its monetary value via a cashier, a kiosk, or other suitable redemption system; (b) a bill dispenser configured to dispense paper currency; (c) a coin dispenser configured to dispense coins or tokens (such as into a coin payout tray); and (d) any suitable combination thereof. In certain embodiments, rather than dispensing bills, coins, or a physical ticket having a monetary value to the user following receipt of an actuation of the cashout device, the payout device is configured to cause a payment to be provided to the user in the form of an electronic funds transfer, such as via a direct deposit into a bank account, a casino account, or a prepaid account of the user; via a transfer of funds onto an electronically recordable identification card or smart card of the user; or via sending a virtual ticket having a monetary value to an electronic device of the user.

In certain embodiments, the at least one output device is a sound generating device controlled by one or more sound cards. In one such embodiment, the sound generating device includes one or more speakers or other sound generating hardware and/or software configured to generate sounds, such as by playing music. In another such embodiment, the gaming establishment device, the component associated with the gaming establishment device, and/or the component of the gaming establishment system provides dynamic sounds coupled with attractive multimedia images displayed on one or more of the display devices to provide an audio-visual representation or to otherwise display full-motion video with sound to attract users to the gaming establishment device, the component associated with the gaming establishment device, and/or the component of the gaming establishment system. In certain embodiments, the gaming establishment device, the component associated with the gaming establishment device, and/or the component of the gaming establishment system displays a sequence of audio and/or visual attraction messages during idle periods to attract potential users to the gaming establishment device, the component associated with the gaming establishment device, and/or the component of the gaming establishment system. The videos may be customized to provide any appropriate information.

The at least one input device may include any suitable device that enables an input signal to be produced and received by the at least one processor of the gaming establishment device, the component associated with the gaming establishment device, and/or the component of the gaming establishment system. In one embodiment, the at least one input device includes a payment device configured to communicate with the at least one processor of the gaming establishment device, the component associated with the gaming establishment device, and/or the component of the gaming establishment system to fund the gaming establishment device. In certain embodiments, the payment device includes zero, one or more of: (a) a bill acceptor into which paper money is inserted; (b) a ticket acceptor into which a ticket or a voucher is inserted; (c) a reader or a validator for credit cards, debit cards, or credit slips into which a credit card, debit card, or credit slip is inserted; (d) a player identification card reader into which a player identification card is inserted; or (e) any suitable combination thereof. In one embodiment, the at least one input device includes a payment device configured to enable the gaming establishment device to be funded via an electronic funds transfer, such as a transfer of funds from a bank account. In another embodiment, the gaming establishment device includes a payment device configured to communicate with a mobile device of a user, such as a mobile phone, a radio frequency identification tag, or any other suitable wired or wireless device, to retrieve relevant information associated with that user to fund the gaming establishment device. When the gaming establishment device is funded, the at least one processor determines the amount of funds entered and displays the corresponding amount.

In various embodiments, the at least one input device includes a plurality of buttons that are programmable by the gaming establishment device, the component associated with the gaming establishment device, and/or the component of the gaming establishment system operator to, when actuated, cause the gaming establishment device, the component associated with the gaming establishment device, and/or the component of the gaming establishment system to perform particular functions. For instance, such buttons may be hard keys, programmable soft keys, or icons icon displayed on a display device of the gaming establishment device, the component associated with the gaming establishment device, and/or the component of the gaming establishment system that are actuatable via a touch screen of the gaming establishment device, the component associated with the gaming establishment device, and/or the component of the gaming establishment system or via use of a suitable input device of the gaming establishment device, the component associated with the gaming establishment device, and/or the component of the gaming establishment system. In certain embodiments, the at least one input device includes a touch-screen coupled to a touch-screen controller or other touch-sensitive display overlay to enable interaction with any images displayed on a display device (as described below). One such input device is a conventional touch-screen button panel. The touch-screen and the touch-screen controller are connected to a video controller. In these embodiments, signals are input to the gaming establishment device, the component associated with the gaming establishment device, and/or the component of the gaming establishment system by touching the touch screen at the appropriate locations.

The at least one wireless communication component includes one or more communication interfaces having different architectures and utilizing a variety of protocols, such as (but not limited to) 802.11 (WiFi); 802.15 (including Bluetooth™); 802.16 (WiMax); 802.22; cellular standards such as CDMA, CDMA2000, and WCDMA; Radio Frequency (e.g., RFID); infrared; and Near Field Magnetic communication protocols. The at least one wireless communication component transmits electrical, electromagnetic, or optical signals that carry digital data streams or analog signals representing various types of information.

The at least one wired/wireless power distribution component includes components or devices that are configured to provide power to other devices. For example, in one embodiment, the at least one power distribution component includes a magnetic induction system that is configured to provide wireless power to one or more user input devices near the gaming establishment device, the component associated with the gaming establishment device, and/or the component of the gaming establishment system. In one embodiment, a user input device docking region is provided, and includes a power distribution component that is configured to recharge a user input device without requiring metal-to-metal contact. In one embodiment, the at least one power distribution component is configured to distribute power to one or more internal components of the gaming establishment device, the component associated with the gaming establishment device, and/or the component of the gaming establishment system, such as one or more rechargeable power sources (e.g., rechargeable batteries) located at the gaming establishment device, the component associated with the gaming establishment device, and/or the component of the gaming establishment system.

In certain embodiments, the at least one sensor includes at least one of: optical sensors, pressure sensors, RF sensors, infrared sensors, image sensors, thermal sensors, and biometric sensors. The at least one sensor may be used for a variety of functions, such as: detecting movements and/or gestures of various objects within a predetermined proximity to the gaming establishment device, the component associated with the gaming establishment device, and/or the component of the gaming establishment system; detecting the presence and/or identity of various persons (e.g., users, casino employees, etc.), devices (e.g., user input devices), and/or systems within a predetermined proximity to the gaming establishment device, the component associated with the gaming establishment device, and/or the component of the gaming establishment system.

The at least one data preservation component is configured to detect or sense one or more events and/or conditions that, for example, may result in damage to the gaming establishment device, the component associated with the gaming establishment device, and/or the component of the gaming establishment system and/or that may result in loss of information associated with the gaming establishment device, the component associated with the gaming establishment device, and/or the component of the gaming establishment system. Additionally, the data preservation system may be operable to initiate one or more appropriate action(s) in response to the detection of such events/conditions.

The at least one motion/gesture analysis and interpretation component is configured to analyze and/or interpret information relating to detected user movements and/or gestures to determine appropriate user input information relating to the detected user movements and/or gestures. For example, in one embodiment, the at least one motion/gesture analysis and interpretation component is configured to perform one or more of the following functions: analyze the detected gross motion or gestures of a user; interpret the user's motion or gestures (e.g., in the context of a casino game being played) to identify instructions or input from the user; utilize the interpreted instructions/input to advance the game state; etc. In other embodiments, at least a portion of these additional functions may be implemented at a remote system or device.

The at least one portable power source enables the gaming establishment device, the component associated with the gaming establishment device, and/or the component of the gaming establishment system to operate in a mobile environment.

The at least one geolocation module is configured to acquire geolocation information from one or more remote sources and use the acquired geolocation information to determine information relating to a relative and/or absolute position of the gaming establishment device, the component associated with the gaming establishment device, and/or the component of the gaming establishment system. For example, in one implementation, the at least one geolocation module is configured to receive GPS signal information for use in determining the position or location of the gaming establishment device, the component associated with the gaming establishment device, and/or the component of the gaming establishment system. In another implementation, the at least one geolocation module is configured to receive multiple wireless signals from multiple remote devices (e.g., the gaming establishment device, the component associated with the gaming establishment device, and/or the component of the gaming establishment system, wireless access points, etc.) and use the signal information to compute position/location information relating to the position or location of the gaming establishment device, the component associated with the gaming establishment device, and/or the component of the gaming establishment system.

The at least one user identification module is configured to determine the identity of the current user or current owner of the gaming establishment device, the component associated with the gaming establishment device, and/or the component of the gaming establishment system. For example, in one embodiment, the current user is required to perform a login process at the gaming establishment device, the component associated with the gaming establishment device, and/or the component of the gaming establishment system in order to access one or more features. Alternatively, the gaming establishment device, the component associated with the gaming establishment device, and/or the component of the gaming establishment system is configured to automatically determine the identity of the current user based on one or more external signals, such as an RFID tag or badge worn by the current user and that provides a wireless signal to the gaming establishment device, the component associated with the gaming establishment device, and/or the component of the gaming establishment system that is used to determine the identity of the current user. In at least one embodiment, various security features are incorporated into the gaming establishment device, the component associated with the gaming establishment device, and/or the component of the gaming establishment system to prevent unauthorized users from accessing confidential or sensitive information.

The at least one information filtering module is configured to perform filtering (e.g., based on specified criteria) of selected information to be displayed at one or more displays of the gaming establishment device, the component associated with the gaming establishment device, and/or the component of the gaming establishment system.

In various embodiments, the gaming establishment device, the component associated with the gaming establishment device, and/or the component of the gaming establishment system includes a plurality of communication ports configured to enable the at least one processor of the gaming establishment device, the component associated with the gaming establishment device, and/or the component of the gaming establishment system to communicate with and to operate with external peripherals, such as: accelerometers, arcade sticks, bar code readers, bill validators, biometric input devices, bonus devices, button panels, card readers, coin dispensers, coin hoppers, display screens or other displays or video sources, expansion buses, information panels, keypads, lights, mass storage devices, microphones, motion sensors, motors, printers, reels, SCSI ports, solenoids, speakers, thumbsticks, ticket readers, touch screens, trackballs, touchpads, wheels, and wireless communication devices.

As generally described above, in certain embodiments, the gaming establishment device, the component associated with the gaming establishment device, and/or the component of the gaming establishment system has a support structure, housing, or cabinet that provides support for a plurality of the input devices and the output devices of the gaming establishment device, the component associated with the gaming establishment device, and/or the component of the gaming establishment system.

It should be appreciated that in certain instances in which the gaming establishment device is an EGM, such an EGM includes certain components and/or are configured to operate in certain manners that differentiate these systems from general purpose computing devices. For instance, EGMs are highly regulated to ensure fairness and, in many cases, EGMs are configured to award monetary awards up to multiple millions of dollars. To satisfy security and regulatory requirements in a gaming environment, hardware and/or software architectures are implemented in EGMs that differ significantly from those of general-purpose computing devices. For purposes of illustration, a description of EGMs relative to general purpose computing devices and some examples of these additional (or different) hardware and/or software architectures found in EGMs are described below.

It should be further appreciated that while one might think that adapting general purpose computing device technologies to the gaming industry and EGMs would be a simple proposition because both general-purpose computing devices and EGMs employ processors that control a variety of devices. However, due to at least: (1) the regulatory requirements placed on EGMs, (2) the harsh environment in which EGMs operate, (3) security requirements, and (4) fault tolerance requirements, adapting general purpose computing device technologies to EGMs can be quite difficult. Further, techniques and methods for solving a problem in the general-purpose computing device industry, such as device compatibility and connectivity issues, might not be adequate in the gaming industry. For instance, a fault or a weakness tolerated in a general purpose computing device, such as security holes in software or frequent crashes, is not tolerated in an EGM because in an EGM these faults can lead to a direct loss of funds from the EGM, such as stolen cash or loss of revenue when the EGM is not operating properly or when the random outcome determination is manipulated.

Certain differences between general purpose computing devices and EGMs are described below. A first difference between EGMs and general-purpose computing devices is that EGMs are state-based systems. A state-based system stores and maintains its current state in a non-volatile memory such that, in the event of a power failure or other malfunction, the state-based system can return to that state when the power is restored or the malfunction is remedied. For instance, for a state-based EGM, if the EGM displays an award for a game of chance but the power to the EGM fails before the EGM provides the award to the player, the EGM stores the pre-power failure state in a non-volatile memory, returns to that state upon restoration of power, and provides the award to the player. This requirement affects the software and hardware design on EGMs. General purpose computing devices are not state-based machines, and a majority of data is usually lost when a malfunction occurs on a general-purpose computing device.

A second difference between EGMs and general-purpose computing devices is that, for regulatory purposes, the software on the EGM utilized to operate the EGM has been designed to be static and monolithic to prevent cheating by the operator of the EGM. For instance, one solution that has been employed in the gaming industry to prevent cheating and to satisfy regulatory requirements has been to manufacture an EGM that can use a proprietary processor (i.e., a master gaming controller) running instructions to provide the game of chance from an EPROM or other form of non-volatile memory. The coding instructions on the EPROM are static (non-changeable) and must be approved by a gaming regulator in a particular jurisdiction and installed in the presence of a person representing the gaming jurisdiction. Any changes to any part of the software required to generate the game of chance, such as adding a new device driver used to operate a device during generation of the game of chance, can require burning a new EPROM approved by the gaming jurisdiction and reinstalling the new EPROM on the EGM in the presence of a gaming regulator. Regardless of whether the EPROM solution is used, to gain approval in most gaming jurisdictions, an EGM must demonstrate sufficient safeguards that prevent an operator or a player of an EGM from manipulating the EGM's hardware and software in a manner that gives him an unfair, and in some cases illegal, advantage.

A third difference between EGMs and general-purpose computing devices is authentication—EGMs storing code are configured to authenticate the code to determine if the code is unaltered before executing the code. If the code has been altered, the EGM prevents the code from being executed. The code authentication requirements in the gaming industry affect both hardware and software designs on EGMs. Certain EGMs use hash functions to authenticate code. For instance, one EGM stores game program code, a hash function, and an authentication hash (which may be encrypted). Before executing the game program code, the EGM hashes the game program code using the hash function to obtain a result hash and compares the result hash to the authentication hash. If the result hash matches the authentication hash, the EGM determines that the game program code is valid and executes the game program code. If the result hash does not match the authentication hash, the EGM determines that the game program code has been altered (i.e., may have been tampered with) and prevents execution of the game program code.

A fourth difference between EGMs and general-purpose computing devices is that EGMs have unique peripheral device requirements that differ from those of a general-purpose computing device, such as peripheral device security requirements not usually addressed by general purpose computing devices. For instance, monetary devices, such as coin dispensers, bill validators, and ticket printers and computing devices that are used to govern the input and output of cash or other items having monetary value (such as tickets) to and from an EGM have security requirements that are not typically addressed in general purpose computing devices. Therefore, many general-purpose computing device techniques and methods developed to facilitate device connectivity and device compatibility do not address the emphasis placed on security in the gaming industry.

To address some of the issues described above, a number of hardware/software components and architectures are utilized in EGMs that are not typically found in general purpose computing devices. These hardware/software components and architectures, as described below in more detail, include but are not limited to watchdog timers, voltage monitoring systems, state-based software architecture and supporting hardware, specialized communication interfaces, security monitoring, and trusted memory.

Certain EGMs use a watchdog timer to provide a software failure detection mechanism. In a normally-operating EGM, the operating software periodically accesses control registers in the watchdog timer subsystem to “re-trigger” the watchdog. Should the operating software fail to access the control registers within a preset timeframe, the watchdog timer will timeout and generate a system reset. Typical watchdog timer circuits include a loadable timeout counter register to enable the operating software to set the timeout interval within a certain range of time. A differentiating feature of some circuits is that the operating software cannot completely disable the function of the watchdog timer. In other words, the watchdog timer always functions from the time power is applied to the board.

Certain EGMs use several power supply voltages to operate portions of the computer circuitry. These can be generated in a central power supply or locally on the computer board. If any of these voltages falls out of the tolerance limits of the circuitry they power, unpredictable operation of the EGM may result. Though most modern general purpose computing devices include voltage monitoring circuitry, these types of circuits only report voltage status to the operating software. Out of tolerance voltages can cause software malfunction, creating a potential uncontrolled condition in the general-purpose computing device. Certain EGMs have power supplies with relatively tighter voltage margins than that required by the operating circuitry. In addition, the voltage monitoring circuitry implemented in certain EGMs typically has two thresholds of control. The first threshold generates a software event that can be detected by the operating software and an error condition then generated. This threshold is triggered when a power supply voltage falls out of the tolerance range of the power supply, but is still within the operating range of the circuitry. The second threshold is set when a power supply voltage falls out of the operating tolerance of the circuitry. In this case, the circuitry generates a reset, halting operation of the EGM.

As described above, certain EGMs are state-based machines. Different functions of the game provided by the EGM (e.g., bet, play, result, points in the graphical presentation, etc.) may be defined as a state. When the EGM moves a game from one state to another, the EGM stores critical data regarding the game software in a custom non-volatile memory subsystem. This ensures that the player's wager and credits are preserved and to minimize potential disputes in the event of a malfunction on the EGM. In general, the EGM does not advance from a first state to a second state until critical information that enables the first state to be reconstructed has been stored. This feature enables the EGM to recover operation to the current state of play in the event of a malfunction, loss of power, etc. that occurred just before the malfunction. In at least one embodiment, the EGM is configured to store such critical information using atomic transactions.

Generally, an atomic operation in computer science refers to a set of operations that can be combined so that they appear to the rest of the system to be a single operation with only two possible outcomes: success or failure. As related to data storage, an atomic transaction may be characterized as series of database operations which either all occur, or all do not occur. A guarantee of atomicity prevents updates to the database occurring only partially, which can result in data corruption.

To ensure the success of atomic transactions relating to critical information to be stored in the EGM memory before a failure event (e.g., malfunction, loss of power, etc.), memory that includes one or more of the following criteria be used: direct memory access capability; data read/write capability which meets or exceeds minimum read/write access characteristics (such as at least 5.08 Mbytes/sec (Read) and/or at least 38.0 Mbytes/sec (Write)). Memory devices that meet or exceed the above criteria may be referred to as “fault-tolerant” memory devices.

Typically, battery-backed RAM devices may be configured to function as fault-tolerant devices according to the above criteria, whereas flash RAM and/or disk drive memory are typically not configurable to function as fault-tolerant devices according to the above criteria. Accordingly, battery-backed RAM devices are typically used to preserve EGM critical data, although other types of non-volatile memory devices may be employed. These memory devices are typically not used in typical general purpose computing devices.

Thus, in at least one embodiment, the EGM is configured to store critical information in fault-tolerant memory (e.g., battery-backed RAM devices) using atomic transactions. Further, in at least one embodiment, the fault-tolerant memory is able to successfully complete all desired atomic transactions (e.g., relating to the storage of EGM critical information) within a time period of 200 milliseconds or less. In at least one embodiment, the time period of 200 milliseconds represents a maximum amount of time for which sufficient power may be available to the various EGM components after a power outage event has occurred at the EGM.

As described previously, the EGM may not advance from a first state to a second state until critical information that enables the first state to be reconstructed has been atomically stored. After the state of the EGM is restored during the play of a game of chance, game play may resume and the game may be completed in a manner that is no different than if the malfunction had not occurred. Thus, for example, when a malfunction occurs during a game of chance, the EGM may be restored to a state in the game of chance just before when the malfunction occurred. The restored state may include metering information and graphical information that was displayed on the EGM in the state before the malfunction. For example, when the malfunction occurs during the play of a card game after the cards have been dealt, the EGM may be restored with the cards that were previously displayed as part of the card game. As another example, a bonus game may be triggered during the play of a game of chance in which a player is required to make a number of selections on a video display screen. When a malfunction has occurred after the player has made one or more selections, the EGM may be restored to a state that shows the graphical presentation just before the malfunction including an indication of selections that have already been made by the player. In general, the EGM may be restored to any state in a plurality of states that occur in the game of chance that occurs while the game of chance is played or to states that occur between the play of a game of chance.

Game history information regarding previous games played such as an amount wagered, the outcome of the game, and the like may also be stored in a non-volatile memory device. The information stored in the non-volatile memory may be detailed enough to reconstruct a portion of the graphical presentation that was previously presented on the EGM and the state of the EGM (e.g., credits) at the time the game of chance was played. The game history information may be utilized in the event of a dispute. For example, a player may decide that in a previous game of chance that they did not receive credit for an award that they believed they won. The game history information may be used to reconstruct the state of the EGM before, during, and/or after the disputed game to demonstrate whether the player was correct or not in the player's assertion.

Another feature of EGMs is that they often include unique interfaces, including serial interfaces, to connect to specific subsystems internal and external to the EGM. The serial devices may have electrical interface requirements that differ from the “standard” EIA serial interfaces provided by general purpose computing devices. These interfaces may include, for example, Fiber Optic Serial, optically coupled serial interfaces, current loop style serial interfaces, etc. In addition, to conserve serial interfaces internally in the EGM, serial devices may be connected in a shared, daisy-chain fashion in which multiple peripheral devices are connected to a single serial channel.

The serial interfaces may be used to transmit information using communication protocols that are unique to the gaming industry. For example, IGT's Netplex is a proprietary communication protocol used for serial communication between EGMs. As another example, SAS is a communication protocol used to transmit information, such as metering information, from an EGM to a remote device. Often SAS is used in conjunction with a player tracking system.

Certain EGMs may alternatively be treated as peripheral devices to a casino communication controller and connected in a shared daisy chain fashion to a single serial interface. In both cases, the peripheral devices are assigned device addresses. If so, the serial controller circuitry must implement a method to generate or detect unique device addresses. General purpose computing device serial ports are not able to do this.

Security monitoring circuits detect intrusion into an EGM by monitoring security switches attached to access doors in the EGM cabinet. Access violations result in suspension of game play and can trigger additional security operations to preserve the current state of game play. These circuits also function when power is off by use of a battery backup. In power-off operation, these circuits continue to monitor the access doors of the EGM. When power is restored, the EGM can determine whether any security violations occurred while power was off, e.g., via software for reading status registers. This can trigger event log entries and further data authentication operations by the EGM software.

Trusted memory devices and/or trusted memory sources are included in an EGM to ensure the authenticity of the software that may be stored on less secure memory subsystems, such as mass storage devices. Trusted memory devices and controlling circuitry are typically designed to not enable modification of the code and data stored in the memory device while the memory device is installed in the EGM. The code and data stored in these devices may include authentication algorithms, random number generators, authentication keys, operating system kernels, etc. The purpose of these trusted memory devices is to provide gaming regulatory authorities a root trusted authority within the computing environment of the EGM that can be tracked and verified as original. This may be accomplished via removal of the trusted memory device from the EGM computer and verification of the secure memory device contents is a separate third-party verification device. Once the trusted memory device is verified as authentic, and based on the approval of the verification algorithms included in the trusted device, the EGM is enabled to verify the authenticity of additional code and data that may be located in the gaming computer assembly, such as code and data stored on hard disk drives.

In at least one embodiment, at least a portion of the trusted memory devices/sources may correspond to memory that cannot easily be altered (e.g., “unalterable memory”) such as EPROMS, PROMS, Bios, Extended Bios, and/or other memory sources that are able to be configured, verified, and/or authenticated (e.g., for authenticity) in a secure and controlled manner.

According to one embodiment, when a trusted information source is in communication with a remote device via a network, the remote device may employ a verification scheme to verify the identity of the trusted information source. For example, the trusted information source and the remote device may exchange information using public and private encryption keys to verify each other's identities. In another embodiment, the remote device and the trusted information source may engage in methods using zero knowledge proofs to authenticate each of their respective identities.

EGMs storing trusted information may utilize apparatuses or methods to detect and prevent tampering. For instance, trusted information stored in a trusted memory device may be encrypted to prevent its misuse. In addition, the trusted memory device may be secured behind a locked door. Further, one or more sensors may be coupled to the memory device to detect tampering with the memory device and provide some record of the tampering. In yet another example, the memory device storing trusted information might be designed to detect tampering attempts and clear or erase itself when an attempt at tampering has been detected.

Mass storage devices used in a general-purpose computing devices typically enable code and data to be read from and written to the mass storage device. In a gaming environment, modification of the gaming code stored on a mass storage device is strictly controlled and would only be enabled under specific maintenance type events with electronic and physical enablers required. Though this level of security could be provided by software, EGMs that include mass storage devices include hardware level mass storage data protection circuitry that operates at the circuit level to monitor attempts to modify data on the mass storage device and will generate both software and hardware error triggers should a data modification be attempted without the proper electronic and physical enablers being present.

It should be appreciated that the terminology used herein is for the purpose of describing particular aspects only and is not intended to be limiting of the disclosure. For example, the singular forms “a”, “an” and “the” are intended to include the plural forms as well, unless the context clearly indicates otherwise. In another example, the terms “including” and “comprising” and variations thereof, when used in this specification, specify the presence of stated features, steps, operations, elements, and/or components, but do not preclude the presence or addition of one or more other features, steps, operations, elements, components, and/or groups thereof. Additionally, a listing of items does not imply that any or all of the items are mutually exclusive nor does a listing of items imply that any or all of the items are collectively exhaustive of anything or in a particular order, unless expressly specified otherwise. Moreover, as used herein, the term “and/or” includes any and all combinations of one or more of the associated listed items. It should be further appreciated that headings of sections provided in this document and the title are for convenience only, and are not to be taken as limiting the disclosure in any way. Furthermore, unless expressly specified otherwise, devices that are in communication with each other need not be in continuous communication with each other and may communicate directly or indirectly through one or more intermediaries.

Various changes and modifications to the present embodiments described herein will be apparent to those skilled in the art. For example, a description of an embodiment with several components in communication with each other does not imply that all such components are required, or that each of the disclosed components must communicate with every other component. On the contrary a variety of optional components are described to illustrate the wide variety of possible embodiments of the present disclosure. As such, these changes and modifications can be made without departing from the spirit and scope of the present subject matter and without diminishing its intended technical scope. It is therefore intended that such changes and modifications be covered by the appended claims.

The invention is claimed as follows:

1. A system comprising:
a processor; and
a memory device that stores a plurality of instructions that, when executed by the processor following an activation of a gaming establishment marker from a first gaming establishment credit system, cause the processor to:
determine gaming establishment marker data associated with the activated gaming establishment marker, and
cause the determined gaming establishment marker data to be written in a block of a permissioned blockchain accessible by a second, different gaming establishment credit system.

2. The system of claim 1, wherein the determined gaming establishment marker data comprises data associated with a user that activated the gaming establishment marker.

3. The system of claim 2, wherein the user is an identified user to the first gaming establishment credit system and an anonymous user to the second, different gaming establishment credit system.

4. The system of claim 2, wherein the memory device stores a plurality of further instructions that, when executed by the processor, cause the processor to enable the user to access the gaming establishment marker data written in the block of the permissioned blockchain.

5. The system of claim 1, wherein the determined gaming establishment marker data comprises data associated with at least one of an amount of funds withdrawn against the activated gaming establishment marker, and when the amount of funds is due.

6. The system of claim 5, wherein the amount of funds accessible by the second, different gaming establishment credit system comprises a range of funds.

7. The system of claim 1, wherein the first gaming establishment credit system is associated with a first entity comprising a first plurality of gaming establishments.

8. The system of claim 1, wherein the memory device stores a plurality of further instructions that, when executed by the processor following a deactivation of the gaming establishment marker, cause the processor to cause data associated with the deactivation of the gaming establishment marker to be written in the block of the permissioned blockchain.

9. A system comprising:
a processor; and
a memory device that stores a plurality of instructions that, when executed by the processor, cause the processor to:
following an activation of a first gaming establishment marker from a first gaming establishment credit system, cause data associated with the first gaming establishment marker to be written in a first block of a permissioned blockchain, and
following an activation of a second, different gaming establishment marker from a second, different gaming establishment credit system, cause data associated with the second, different gaming establishment marker to be written in a second block of the permissioned blockchain.

10. The system of claim 9, wherein the data associated with the first gaming establishment marker comprises user data.

11. The system of claim 10, wherein the user data associated with the first gaming establishment marker comprises an identified user to the first gaming establishment credit system and an anonymous user to the second, different gaming establishment credit system.

12. The system of claim 9, wherein the memory device stores a plurality of further instructions that, when executed by the processor following a deactivation of the first gaming establishment marker, cause the processor to cause data associated with the deactivation of the first gaming establishment marker to be written in the first block of the permissioned blockchain.

13. A method of operating a system, the method comprising:
following an activation of a gaming establishment marker from a first gaming establishment credit system:
determining, by a processor, gaming establishment marker data associated with the activated gaming establishment marker, and
causing, by the processor, the determined gaming establishment marker data to be written in a block of a permissioned blockchain accessible by a second, different gaming establishment credit system.

14. The method of claim 13, wherein the determined gaming establishment marker data comprises data associated with a user that activated the gaming establishment marker.

15. The method of claim 14, wherein the user is an identified user to the first gaming establishment credit system and an anonymous user to the second, different gaming establishment credit system.

16. The method of claim 14, further comprising enabling the user to access the gaming establishment marker data written in the block of the permissioned blockchain.

17. The method of claim 13, wherein the determined gaming establishment marker data comprises data associated with at least one of an amount of funds withdrawn against the activated gaming establishment marker, and when the amount of funds is due.

18. The method of claim 17, wherein the amount of funds accessible by the second, different gaming establishment credit system comprises a range of funds.

19. The method of claim 13, wherein the first gaming establishment credit system is associated with a first entity comprising a first plurality of gaming establishments.

20. The method of claim 13, further comprising, following a deactivation of the gaming establishment marker, causing, by the processor, data associated with the deactivation of the gaming establishment marker to be written in the block of the permissioned blockchain.
